(19) United States
(12) Patent Application Publication
(10) Pub. No.: US 2012/0258540 A1
(43) Pub. Date: Oct. 11, 2012

(54) METHODS FOR MODIFYING VIRUS SURFACES

(75) Inventors: Joseph M. LeDoux, Decatur, GA (US); Louis A. Lyon, Mariettta, GA (US); Nimisha Gupta, Mechanicsville, VA (US)

(73) Assignee: GEORGIA TECH RESEARCH CORPORATION, Atlanta, GA (US)

(21) Appl. No.: 12/514,353

(22) PCT Filed: Nov. 13, 2007
(86) PCT No.: PCT/US2007/084552

§ 371 Date: Jan. 14, 2010

Related U.S. Application Data
(60) Provisional application No. 60/858,575, filed on Nov. 13, 2006.

(51) Int. Cl. C12N 15/867; C12N 7/00

(52) U.S. Cl. 435/456; 435/320.1; 435/235.1

(57) ABSTRACT

Nucleic acid delivery vehicles and methods of their use are provided. One embodiment provides a virus having a lipid-polymer conjugate intercalated into the virus's membrane. The lipid-polymer conjugate includes a biocompatible polymer having first and second ends, a lipid conjugated to the first end, and a targeting moiety conjugated to the second end. The lipid is preferably a multi-chain lipid. The virus encodes one or more polypeptides that can help reduce or mitigate one or more symptoms of a disease or pathology. The lipid-polymer conjugate advantageously reduces non-specific binding of the virus while the targeting moiety enhances binding to specific cells or tissues.

### CROSS-REFERENCE TO RELATED APPLICATIONS

This application claims priority to and benefit of U.S. Provisional Patent Application No. 60/858,575 filed on Nov. 13, 2006, and where permissible is incorporated by reference in its entirety.

### FIELD OF THE INVENTION

Aspects of the invention are generally related to gene transfer and methods of modifying nucleic acid delivery vehicles to improve delivery of nucleic acids.

### BACKGROUND OF THE INVENTION

Gene therapy is a promising approach to the treatment of disease. Unfortunately, to date there have been few successes in clinical settings, primarily due to the many shortcomings of the current generation of gene transfer technologies. One major shortcoming of virtually all gene transfer vectors is the inability to strictly control their tropism, the types of cells to which they are able to transfer genes. For many applications, particularly for most in vivo gene therapies, the tropism of gene transfer vectors needs to be narrowed so that genes are transferred only to the cells and tissues of interest and to no others. For other applications, such as in cystic fibrosis gene therapy, the tropism of gene transfer vectors needs to be expanded so that genes can be transferred to the cell types of interest.

Three major approaches have been taken to modify the tropism of retroviruses, each of which involves the modification or replacement of the viral envelope proteins. One common approach has been to genetically engineer the envelope proteins of the virus to contain a domain that will bind to a receptor that is specific for the cell type of interest. For example, in one study a portion of the N-terminus of the ecotropic envelope protein was replaced with sequences encoding for the polypeptide hormone erythropoietin (EPO) (Kasahara, et al., Science, 266(5189):1373-6 (1994)). Remarkably, the resulting ecotropic retrovirus, which normally cannot infect human cells, was able to transfer genes specifically to human cells that expressed the EPO receptor. Similar results have been found by others (Liu et al., 2000). Studies such as these were noteworthy because they proved that it is possible to engineer cell-type specific retroviruses. Unfortunately, the gene transfer efficiency of these viruses was much lower than that of wild-type viruses, and too low to be of practical use in human gene therapy protocols (Cosset, et al., J. Vivol., 69(10):6314-22 (1995); Kasahara, et al., Science, 266(5189):1373-6 (1994); Krishna, et al., Biotechnol. Prog., 21(1):263-73 (2005); Somia, et al., Proc. Natl. Acad. Sci. U.S.A., 92(16):7570-4 (1995); Valsesia-Wittmann, et al., J. Vivol., 70(3):2059-64 (1996)).

Another approach to alter the tropism of retroviruses is to form pseudotyped viruses (Chen, et al., Proc. Natl. Acad. Sci. U.S.A., 93(19):10057-62 (1996); Jung, et al., Biotechnol. Prog., 20(6):1810-6 (2004); Reiser, Gene Ther., 7(10:910-3 (2000)). Pseudotyped retroviruses are usually composed of material from two viruses: envelope proteins from the virus with the desired tropism, and a retrovirus core that carries with it the desired gene transfer functions. Frequently, the envelope proteins are not efficiently incorporated into the lipid bilayers of the retrovirus cores, or if they are incorporated, fail to function properly. Sometimes it is possible to rescue the infectivity of the pseudotyped virus by genetically engineering the envelope proteins to be more efficiently incorporated into the particles, but this is essentially a trial-and-error process since the mechanism by which envelope proteins are incorporated or excluded from retroviruses is not well-understood (Hohne, et al., Virology, 261(1):70-8 (1999); Indraccolo, et al., Gene Ther., 5(2):209-17 (1998); Kobinger, et al., Nat. Biotechnol., 19(3):225-30 (2001); Wool-Lewis and Bates, J. Vivol., 72(4):3155-60 (1998)). Pseudotyping can be an effective method for changing or broadening the tropism of retroviruses, but it is rarely an effective means to create a targeted retrovirus, one with a narrow tropism that is restricted to one cell type.

Covalent modification of the envelope proteins of retroviruses has also been used to alter the tropism of recombinant retroviruses. In this approach, viruses that do not normally infect human cells have been chemically modified to broaden their tropism to include human cell types. For example, Neda et al covalently coupled lactose to ecotropic retroviruses, which normally can only infect rodent cells, to enable them to transduce a human hepatoma cell line that expresses a receptor that binds lactose (Neda et al., J. Biol. Chem., 266(22):14143-6 (1991)). Unfortunately, the levels of gene transfer were very low. Similar results were found when retroviruses were modified with other functional groups (Gollan and Green, J. Virol., 76(7):3558-63 (2002); Reddy, et al., J. Control. Release, 74(1-3):77-82 (2001); Zhong, et al., J Virol, 75(21):10393-400 (2001)). These studies show that covalent modification can be used to alter the tropism of viruses but often results in gene transfer levels that are too low to be of any therapeutic use.

Each of these three methods sought to alter viral tropism by influencing the initial event in virus infection: binding of the virus to the cell. By changing the nature of the interactions between the viral envelope protein and its cognate cellular receptor, it was hoped that virus binding to cells could be controlled. Recent work suggests that these approaches may be flawed because retroviruses appear to bind to cells via interactions that are independent of the envelope protein (Davis, et al., Biophys. Chem., 97(2-3):159-72 (2002); Pizzato, et al., Gene Ther., 8(14):1088-96 (2001)). For example, one group showed that murine leukemia retroviruses lacking surface envelope proteins (‘bald’ viruses) bind to TE671 cells just as avidly as retroviruses that are pseudotyped with the amphotropic or ecotropic envelope protein (Pizzato, et al., Gene Ther., 8(14):1088-96 (2001)). Another group showed that amphotropic retroviruses bind equally well to receptor-negative and receptor-positive CHO cells (Davis, et al., Biophys. Chem., 97(2-3):159-72 (2002)). These studies demonstrate that retrovirus binding is controlled by factors other than their viral envelope proteins, and that these proteins are not, as previously thought, required for virus binding, but are primarily used for inducing fusion and entry of the virus particle after the virus binds to the cell. Clues to what mediates these early receptor-independent virus-cell binding interactions have come from studies of the composition of the lipid bilayer of retroviruses, which is derived from the plasma membrane of the cells that produced them. The protein composition of these lipid bilayers is similar to that of the plasma membrane, and includes a number of cellular proteins, including integrins and other molecules that play important roles in cell adhesion (Cantin, et al., J. Virol., 79(11):6577-87 (2005); Liao, et al., AIDS Res. Hum. Retroviruses, 16(4):355-66 (2000)). Most likely, some of these proteins mediate the initial binding events between retroviruses and cells.

Several patents describe various methods of modifying viruses to increase the efficiency of nucleic acid delivery and regulate tropism. U.S. Pat. No. 6,569,426 discloses modifying a virus using polyethylene glycol (PEG) while retaining virus infectivity. The patent also discloses covalently and noncovalently attaching PEG to the virus surface. The patent also discloses linking the PEG to the surface of the virus.

U.S. Published Patent Application No. 2003/0180261 also discloses a method of virus modification using PEG in which the virus infectivity is retained. The application specifies the molecular weight of the PEG molecules for effective virus attachment.

U.S. Published Patent Application No. 2002/0034498 describes modifying virus using PEG without affecting viral infectivity by using an intermediate antibody as a linker between the virus and PEG.

Lipid-PEG conjugates can be used for delivering plasmid DNA intracellularly. For example U.S. Pat. No. 6,852,334, U.S. Pat. No. 6,562,371 and U.S. Published Patent Application No. 2003/0211142 describe specific membrane components on spherical lipid particles used to deliver drugs. In some instances the particles are coated with PEG molecules to mitigate an in vivo immune response.

U.S. Pat. No. 6,852,334 describes a tripartite system consisting of a lipid moiety, a hydrophilic polymer and a polycationic moiety as a method of delivering plasmids to cells. The process by which this construct would transfer genetic material to cells is known as transfection, which is completely different from transduction. The '334 patent does not disclose or suggest a system for transducing cells, i.e, the transfer of genetic material (and its phenotypic expression) to a cell by a virus. U.S. Pat. No. 6,562,371 describes a tripartite system for delivering nucleic acids to cells. The patent discloses that the liposome constituent chemistry has increased stability in the blood. The technology was to be used in renal diseases, which are accompanied by a large production of proteoglycans in the injured portion of the tissue or organ.

Furthermore, several issued patents discuss conjugates of PEG molecules tethered to a ligand for the purpose of cell receptor targeting. See for example U.S. published Patent Application No. 2003/0124742 and U.S. Pat. No. 5,620,689. U.S. Pat. No. 5,620,689 discusses the use of PEG coated liposomes which have a target antibody on one end of the PEG molecule in order to increase targeting of liposomes to specific cells or tissues.

U.S. published Patent App. No. 2003/0124742 describes a tripartite system including a water-soluble biocompatible polymer, one or more spacer peptides having a chemical agent that would be released and bound to another spacer peptide, and a targeting peptide linked to the polymer.

None of the cited patents or patent applications disclose a rapid and flexible method for altering the surface of viruses to control tropism and increase transduction efficiency.

Thus, it is an object of the invention to provide compositions and methods for controlling virus tropism.

It is another object to provide methods for altering the surface of viruses to control tropism.

It is still another object to provide methods for improving or increasing gene transfer from a virus to a cell.

It is another object to provide improved methods for gene therapy.

### SUMMARY OF THE INVENTION

Nucleic acid delivery vehicles and methods of their use are provided. One embodiment provides a virus having a lipid-polymer conjugate intercalated into the virus's membrane. The lipid-polymer conjugate includes a biocompatible polymer having first and second ends, a lipid conjugated to the first end, and a targeting moiety conjugated to the second end. The lipid is preferably a multi-chain lipid. The virus genetic material is designed to reduce or mitigate one or more symptoms of a disease or pathology, and may encode, for example, one or more therapeutic polypeptides or short hairpin RNA (shRNA) molecules, siRNA, micro RNA or a combination thereof. The lipid-polymer conjugate advantageously reduces non-specific binding of the virus while the targeting moiety enhances binding to specific cells or tissues.

Another embodiment provides a method for modifying the surface of a virus by contacting the virus with a lipid-polymer conjugate such that the lipid polymer conjugate intercalates into the lipid bilayer of the virus. Lipid component of the lipid-polymer construct facilitates insertion of the conjugate into the lipid bilayer of the virus, the polymer helps reduce non-specific binding of the virus.

Still another embodiment provides a method for detecting or imagining a virus by contacting the virus with the disclosed lipid-polymer conjugates wherein the lipid-polymer conjugate includes a detectable label, for example a fluorescent dye.

Yet another embodiment provides a vaccine. The vaccine includes a retrovirus encoding an immunogenic polypeptide or an inhibitory shRNA. The retrovirus also includes one or more of the disclosed lipid-polymer conjugates intercalated into the membrane of the retrovirus. One or more of the lipid-polymer conjugates could function to target the virus to bind to a specific cell type of the immune system, such as dendritic cells. In addition, one or more of the lipid-polymer conjugates could serve as adjuvants, increasing the immunogenicity of the virus particles themselves, causing them to stimulate the cells to which they bind.

Kits containing the disclosed nucleic acid delivery vehicles or components thereof are also provided.

### BRIEF DESCRIPTION OF THE DRAWINGS

FIG. 1 shows the structure of a representative lipid-polymer conjugate used to modify the viruses, DSPE-PEG(2000)Amine 1,2-Distearoyl-sn-Glycero-3-Phosphoethanolamine-N-[Amino(Polyethylene Glycol)2000] (Ammonium Salt). The conjugate was from Avanti Polar Lipids (MW=3461 Da).

FIG. 2A is a bar graph of ng/L biotin in pellets of virus incubated with DSPE-PEG(2000)-biotin compared to a control. FIG. 2B is a bar graph of concentration of p30 (OD 490 nm) in the original virus stocks (BEFORE), decanted supernatant (SN), and the resuspended pellets (Pellet).

FIG. 3A is a bar graph of virus-associated biotin (% maximum) in virus particles incubated with DSPE-PEG(2000)-biotin and resuspended in TBS w/ or w/o 10% bovine calf serum (BCS). FIG. 3B is a panel of bar graphs of biotin (ng/L) in pellets allowed to desorb in TBS (VL TBS) or TBS with serum (VL TBS ser).

FIG. 4 is a panel of bar graphs of biotin (ng/L) in virus particles incubated for the indicated time.

FIG. 5 is a line graph of p30 concentration (OD 490 nm) and (ng/ml) at the indicated incubation times.

FIG. 6 is a bar graph showing bound fraction of retrovirus modified using lipid-PEG-biotin for modified (V+L+PB) or unmodified (V+PB, v only) virus incubated onto strepavidin coated plates.

FIG. 7 is a bar graph showing titer (colonies/ml) for cells transduced with modified or unmodified amphotropic lentivirus.

### DETAILED DESCRIPTION OF THE INVENTION

### I. Definitions

The term “targeting moiety” refers to substances that direct the nucleic acid delivery vehicle to a specific cell, organelle, or tissue.

The term “lipid” refers to fatty acids and their derivatives, and substances related biosynthetically or functionally to these compounds.

In describing and claiming the disclosed subject matter, the following terminology will be used in accordance with the definitions set forth below.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Praline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides in the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gin), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M, and Griffin, H. G., Eds., Humana Press, New Jersey, 1994; Sequence Analysis in Molecular Biology, von Heinje, G., Academic Press, 1987; and Sequence Analysis Primer, Gribskov, M and Devereux, J., Eds., M Stockton Press, New York, 1991; and Carillo, H., and Lipman, D., SIAM J Applied Math., 48: 1073 (1988)).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “low stringency” refers to conditions that permit a polynucleotide or polypeptide to bind to another substance with little or no sequence specificity.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “treating” includes alleviating the symptoms associated with a specific disorder or condition and/or preventing or eliminating said symptoms.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

The term “targeting moiety” refers to a signal that directs a molecule to a specific cell, tissue, organelle, or intracellular region. The signal can be polynucleotide, polypeptide, or carbohydrate moiety or can be an organic or inorganic compound sufficient to direct an attached molecule to a desired location. Exemplary targeting signals include cell targeting signals known in the art such as those provided in Table 1 and described in Wagner et al., Targeting of Polyplexes: Toward Synthetic Virus Vector Systems (Adv in Gen, 53:2005, 333-354) the disclosures of which are incorporated herein by reference in their entirety. It will be appreciated that the entire sequence listed in Table 1 need not be included, and modifications including truncations of these sequences are within the scope of the disclosure provided the sequences operate to direct a linked molecule to a specific cell type. Targeting signals of the present disclosure can have 80 to 100% identity to the sequences in Table 1. One class of suitable targeting signals include those that do not interact with the targeted cell in a receptor:ligand mechanism. For example, targeting signals include signals having or conferring a net charge, for example a positive charge. Positively charged signals can be used to target negatively charged cell types such as neurons and muscle. Negatively charged signals can be used to target positively charged cells.

TABLE 1
Targeting Moieties.

| Cell Surface Antigen/Cell Type | Cell Ligand |
|---|---|
| Airway cells | Surfactant proteins A and B |
| Arterial wall | Artery wall binding peptide |
| ASGP receptor | Asialoglycoproteins |
| ASGP receptor | Synthetic galactosylated ligands |
| Carbohydrates | Lectins |
| CD3 | Anti-CD 3 |
| CD5 | Anti-CD 5 |
| CD44 | hyaluronic acid fragments |
| CD117 | Steel factor, Anti CD117 |
| EGF-R | EGF, EGF peptide Anti EGF-R, |
| | TGF-alpha |
| ErbB2 | anti ErbB2 |
| FcR | IgG |
| FGF2-R | basic FGF |
| Folate receptor | Folate |
| Hepatocyte basolateral surface | Malarial circumsporozoite protein |
| Her2 | Anti HER2 |
| Insulin receptor | Insulin |
| Integrin | RGD peptide |
| LDL receptor family (hepatocytes) | Receptor associated protein (RAP) |
| Mannose receptor (macrophages) | Synthetic ligands, mannosylated |
| Nerve growth factor (NGF) receptor | NGF serived synthetic peptide |
| TrkA | |
| Neuroblastoma | Antibody ChCE7 |
| Ovarian carcinoma cell surface | Antibody OV-TL16 Fab′ fragment |
| antigen OA3 | |
| PECAM (lung endothelium) | anti-PECAM antibody |
| Poly-immunoglobulin receptor | Anti-secretory component |
| Serpin-enzyme receptor | peptide ligand |
| Surface immunoglobulin | Anti-IgG, Anti-idiotype |
| Thrombomodulin | Anti-thrombomodulin |
| Tn carbohydrate | Anti-Tn |
| Transferrin receptor | Transferrin |
| Airway cells | Surfactant proteins A and B |
| Arterial wall | Artery wall binding peptide |
| ASGP receptor | Asialoglycoproteins |
| ASGP receptor | Synthetic galactosylated ligands |
| Carbohydrates | Lectins |

“Tropism” refers to the capacity of viruses to infect discrete populations of cells within an organism, tissue, or tissue culture dish. The tropism of a virus is influenced by the interaction between a variety of host and viral factors. Tropism is controlled to a large extent by the viral cell attachment proteins (i.e., viral envelope proteins) and their cognate cellular receptors. However, the mere presence of a functional viral receptor is not always sufficient to allow viral infection of the target cells. Post-binding steps of infection, such as virus fusion and intracellular trafficking, can also be important determinants of tropism. The propensity of a gene transfer vector, such as a virus, to bind to a specific cell type, tissue, or organ, and to successfully negotiate post-binding steps of gene delivery, can be accomplished by means of functional moieties that are coupled to the genetic material or virus. These functional moieties could include peptides or other molecules that have a high binding affinity for a cell surface marker or protein that is specifically expressed by the targeted cell type. Alternatively, the functional moieties could include peptides or other molecules with functions other than binding, such as to increase the fusogenicity of the vector or virus to enhance their ability to enter the cytosol of the cell, or to control the intracellular trafficking itinerary of the vector or virus to maximize the efficiency with which the genetic material reaches the nucleus.

As used herein, the term “exogenous DNA” or “exogenous nucleic acid sequence” or “exogenous polynucleotide” refers to a nucleic acid sequence that was introduced into a cell or organelle from an external source. Typically the introduced exogenous sequence is a recombinant sequence.

As used herein, the term “transfection” refers to the introduction of a nucleic acid sequence into the interior of a membrane enclosed space of a living cell, including introduction of the nucleic acid sequence into the cytosol of a cell as well as the interior space of a mitochondria, nucleus or chloroplast. The nucleic acid may be in the form of naked DNA or RNA, associated with various proteins or the nucleic acid may be incorporated into a vector.

As used herein, the term “transduction” refers to transfer of genetic material to a cell by a virus.

As used herein, the term “vector” is used in reference to a vehicle used to introduce a nucleic acid sequence into a cell. A viral vector is virus that has been modified to allow recombinant DNA sequences to be introduced into host cells or cell organelles.

As used herein, the term “polynucleotide” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term “nucleic acid” or “nucleic acid sequence” also encompasses a polynucleotide as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

“Oligonucleotide(s)” refers to relatively short polynucleotides. Often the term refers to single-stranded deoxyribonucleotides, but it can refer as well to single- or double-stranded ribonucleotides, RNA:DNA hybrids and double-stranded DNAs, among others.

### II. Nucleic Acid Delivery Vehicles

Compositions and methods for delivering nucleic acids to a target cell or tissue are provided. In certain embodiments, the disclosed compositions control viral tropism at the level of binding, but do so without altering the function of the viral envelope proteins. Viral envelope proteins are needed for post-binding steps of infection (fusion and entry) and are difficult to modify without inactivating them. The surface properties of a virus are modified by anchoring lipid-polymer conjugates within the virus's lipid bilayers or membranes. The lipid-polymer conjugates reduce or eliminate non-specific binding interactions between the viruses and cells.

One embodiment provides a method for increasing gene transfer efficiency and selectivity by non-covalently modifying a virus with a lipid-polymer conjugate having one or more targeting moieties that enhance the selectivity and binding to target cells or tissue. Another embodiment provides a system for assembling functional groups onto the surface of virus particles that reduce non-specific binding and enhance specific binding without reducing or eliminating the ability of the viral envelope proteins to interact with their receptors and mediate fusion between the virus and cell. Preferred lipid-polymer conjugates are those that rapidly and stably intercalate within the lipid bilayer of retroviruses without significantly reducing viral infectivity. In one embodiment, the conjugates anchor functional groups within the lipid bilayer of retroviruses that prevent the viruses from binding to innocent bystander cells while enhancing the specific binding of the virus to cells that express a targeted receptor.

A representative nucleic acid delivery vehicle includes a virus encoding one or more polypeptides. Preferably the polypeptides are selected to be expressed in a target cell or tissue once the virus transduces the cell or tissue. The virus includes a lipid-conjugated polymer intercalated in the virus's lipid bilayer. The lipid-conjugated polymer includes a lipid, a biocompatible polymer and targeting moiety.

A. Virus

In one preferred embodiment, the nucleic acid delivery vehicle includes a retrovirus. Retroviruses used for gene transfer studies are often derived from the Moloney murine leukemia virus (MLV) or from the human immunodeficiency virus type 1 (HIV-1). The most significant functional difference between these two retroviruses is that MLV viruses can only infect cells that are actively dividing, whereas viral vectors derived from HIV can infect cells even if they are not dividing, an important advantage for many in vivo gene therapy protocols. The type of cells retroviruses are able to infect (i.e., their tropism) is largely determined by the interaction between envelope proteins that protrude from the surface of the virus and virus receptors on the surface of the cell (De Larco, et al., Int. J. Cancer, 21(3):356-60 (1978); Kavanaugh, et al., Proc. Natl. Acad. Sci. U.S.A., 91(15):7071-5 (1994)). Frequently, retroviruses that are being studied for use in human gene therapy have had their wild-type envelope protein replaced with one from another virus to form a pseudotyped virus (i.e., a virus composed of proteins from more than one virus). The most commonly used envelope proteins for pseudotyping retroviruses are the amphotropic and VSVG envelope proteins (Chen, et al., Proc. Natl. Acad. Sci. U.S.A., 93(19):10057-62 (1996); Kavanaugh, et al., Proc. Natl. Acad. Sci. USA., 91(15):7071-5 (1994)). These proteins are favored primarily because they enable the viruses to transduce a wide range of human cell types. Their lack of cell-type specificity can be a liability when the viruses must be delivered directly to the patient, in vivo, as is required when the cells or tissues that are being treated cannot be removed from the patient (e.g., brain, heart, lungs). In addition, despite their ability to transfer genes to a wide range of human cell-types, there are still a number of clinically relevant cell types that cannot be transduced by VSVG or amphotropic pseudotyped retroviruses (Johnson, et al., Gene Ther., 7(7):568-74 (2000); Wang, et al., Curr. Opin. Mol. Ther., 2(5):497-506 (2000)).

It will be appreciated that the disclosed lipid conjugates can be used with any enveloped virus—that is, any virus that has a lipid bilayer. The virus to be used can be selected based on the application of the technology. For example, the technology can be used for three major applications: 1) gene transfer, 2) labeling and detection of viruses, and 3) vaccines (see below). Suitable viruses include, but are not limited to recombinant retroviruses derived from murine leukemia viruses (MuLV). These are also called ‘oncogenic retroviruses’. They can be ‘pseudotyped’ with a number of different envelope proteins, including amphotropic, ecotropic, 10A1, GALV, and VSV-G. These viruses are called “recombinant MuLV amphotropic retroviruses”. Recombinant lentiviruses derived from HIV-1 can also be used. Again, these can be pseudotyped with a number of different envelope proteins, including amphotropic, ecotropic, 10A1, and VSV-G. Lentiviruses can also be derived from simian immunodeficiency viruses (SIV) and bovine immunodeficiency viruses (BIV). Other enveloped viruses that can be used include Spumaviruses (e.g., human foamy viruses, simian foamy viruses), Herpes simplex virus, Flaviviruses (eg., Tickborne encephalitis viruses, Yellow fever), Paramyxoviridae (human paramyxovirus, measles, newcastle disease virus), Vaccinia virus, Togaviridae (alphavirus, semliki forest virus), Viral hemorrhagic fevers (filoviruses [e.g., Ebola, Marburg] and arenaviruses [e.g., Lassa, Machupo]), Nipah virus, viral encephalitis (alphaviruses [e.g., venezuelan equine encephalitis, eastern equine encephalitis, western equine encephalitis]), Bunyaviridae (e.g, Hantaviruses), Influenza, and Japanese enchepalitis virus.

B. Lipid Component

Lipid component of the disclosed conjugates can be any lipid or hydrophobic chain that is capable of intercalating into the virus membrane. Preferred lipids include, but are not limited to double chained lipids for example, 1,2-distearoyl-sn-glycero-3-phosphoethanolamine-N-[methoxy(polyethylene glycol)-2000], dimyristoyl, dimyristoyl, dioleoyl, myristoyl, oleoly, and linoleoyl. In one embodiment the lipid is cholesterol.

In certain embodiments, the nucleic acid delivery compositions of the present invention may comprise one or more lipids. A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. Of course, compounds other than those specifically described herein that are understood by one of skill in the art as lipids are also encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof.

1. Lipid Types

A neutral fat may comprise a glycerol and a fatty acid. A typical glycerol is a three carbon alcohol. A fatty acid generally is a molecule comprising a carbon chain with an acidic moeity (e.g., carboxylic acid) at an end of the chain. The carbon chain may of a fatty acid may be of any length, however, it is preferred that the length of the carbon chain be of from about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, to about 30 or more carbon atoms, and any range derivable therein. However, a preferred range is from about 14 to about 24 carbon atoms in the chain portion of the fatty acid, with about 16 to about 18 carbon atoms being particularly preferred in certain embodiments. In certain embodiments the fatty acid carbon chain may have an odd number of carbon atoms, however, an even number of carbon atoms in the chain may be preferred in certain embodiments. A fatty acid having only single bonds in its carbon chain is called saturated, while a fatty acid comprising at least one double bond in its chain is called unsaturated.

Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride includes a glycerol and one fatty acid, a diglyceride comprises a glycerol and two fatty acids, and a triglyceride comprises a glycerol and three fatty acids.

A phospholipid generally includes either glycerol or a sphingosine moeity, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phopholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphatidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.k.a cardiolipin), an egg phosphatidylcholine, a dipalmitoyl phosphatidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but includes a carbohydrate group rather than a phosphate group attached to a primary hydroxyl group of the sphingosine. A type of glycolipid called a cerebroside includes one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Vitamin D is another example of a sterol, and is involved in calcium absorption from the intestine.

Cholesterol is another example of a steroid, and generally serves structural rather than regulatory functions. Cholesterol is present in the plasma membrane of cells and in the lipid bilayer of retroviruses, lentiviruses, and other enveloped viruses. Cholesterol in both cellular and viral membranes appears to play a critical role in virus transduction. It is contemplated that the use of cholesterol and/or its derivatives as the lipid component of the lipid-polymer conjugates, may increase their incorporation into the viral particles, and may increase the efficiency of vector and viral-mediated nucleic acid delivery.

A terpene is a lipid having one or more five carbon isoprene groups. Terpenes have various biological functions, and include, for example, vitamin A, coenyzme Q and carotenoids (e.g., lycopene and beta-carotene).

2. Making Lipids

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, suitable lipids can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). In certain embodiments, stock solutions of lipids in chloroform or chloroform/methanol can be stored at about −20° C. Preferably, chloroform is used as the only solvent since it is more readily evaporated than methanol.

C. Biocompatible Polymer

Any water-soluble biocompatible polymer can be used. Exemplary water-soluble biocompatible polymers include but are not limited to polyalkylene oxides, e.g. polyethylene oxide. Suitable polyalkylene oxides include a member selected from the group consisting of polyethylene oxides, alpha-substituted polyalkylene oxide derivatives, polyethylene glycol homopolymers and derivatives thereof, polypropylene glycol homopolymers and derivatives thereof, alkyl-capped polyethylene oxides, bis-polyethylene oxides, copolymers of poly(alkylene oxides), branched polyethylene glycols, star polyethylene glycols, pendant polyethylene glycols, block copolymers of poly(alkylene oxides) and activated derivatives thereof. In a further embodiment, the polyalkylene oxide is an alkyl blocked pendant polyethylene glycol (“pPEG”) or mono-methyl blocked pendant polyethylene glycol (“mpPEG”).

The use of multi-arm, dendritic, or star-type polyalkylene glycols is advantageous. The multiple pendant groups on the polymer permit the attachment of multiple agents to the conjugate, to improve efficacy of the conjugate. For example, the polymer may include 2, 3, 4, 5, 6, 7, 8, or 9 or more molecules of an agent such as a targeting moiety. In one aspect, the polymer includes at least 3, at least 4, at least 5 or at least 6 molecules of the agent.

Additionally, although the use of acyl blocked pendant polyalkylene glycols has similar advantages to the use of alkyl blocked pendant polyalkylene glycols, the use of a diacyl blocked pendant polyalkylene glycol, such as bis-hemisuccinyl pendant polyethylene glycol or monomethyl-hemisuccinyl pendant polyethylene glycol offers the advantage that additional reactive carboxyl group(s) are introduced which can be further derivatized. This is a particular advantage when the pendant groups contain carboxyl moieties, since the possibility of differential reactivity between the hemisuccinyl carboxyl groups and the pendant carboxyl groups is created.

A preferred polymer is PEG. PEG is a neutral, water-soluble, nontoxic, non-adhesive polymer that has been frequently used to increase the bioavailability, stability, and circulation times of liposomes and protein-based pharmaceuticals (Janssen, et al., Int. J. Pharm., 254(1):55-8 (2003); Otsuka, et al., Adv. Drug Deliv. Rev., 55(3):403-19 (2003)), and has been used to reduce the level of inflammation and improve the pharmacokinetics of recombinant adenoviruses when they are injected into patients (Croyle, et al., J. Virol., 75(10):4792-801 (2001); Ogawara, et al., Hum. Gene Ther., 15(5):433-43 (2004)).

Exemplary synthetic hydrophobic polymers suitable for use in the lipid-polymer conjugate include polypropylene oxide, polyethylene, polypropylene, polycarbonate, polystyrene, polysulfone, polyphenylene oxide and polytetramethylene ether. The molecular weight of the hydrophobic polymer is a key design factor that must be optimized for the particular function of the lipid-polymer conjugate. For example, lipid-polymer conjugates that are designed to minimize or prevent non-specific binding of the vector or virus to cells will most likely benefit from the use of high molecular weight hydrophobic polymers (i.e., greater than 20,000 daltons). In contrast, lipid-polymer conjugates that are designed to enhance binding or fusion of the vector or virus to a specific cell type will most likely benefit most from the use of lower molecular weight hydrophobic polymers (e.g., 2,000 to 5,000 daltons).

D. Targeting Moiety

In certain embodiments, nucleic acid delivery vehicles include at least one targeting moiety to an organelle, cell, tissue, organ or organism. Any targeting agent described herein or known to one of ordinary skill in the art may be used in the compositions and methods, either alone in combination with other targeting moieties or agents. In specific embodiments, the targeting agent is attached to the biocompatible polymer so that the targeting moiety is on the surface of the virus.

Various agents for targeting molecules to specific cells, tissue, organs and organisms are known to those of ordinary skill in the art, and may be used in the methods and compositions of the present invention. In certain embodiments, for example, targeting agents may include, but are not limited to, EGF, transferrin, an anti-prostate specific membrane antigen antibody, endothelial specific peptides and bone specific ligands.

In another non-limiting example, a targeting moiety may include an antibody, cytokine, growth factor, hormone, lymphokine, receptor protein, such as, for example CD4, CD8 or soluble fragments thereof, a nucleic acid which binds corresponding nucleic acids through base pair complementarity, or a combination thereof (U.S. Pat. No. 6,071,533, incorporated herein by reference). In other embodiments, the targeting moiety may include a cellular receptor-targeting moiety, a fusogenic ligand, a nucleus targeting ligand, or a combination thereof (U.S. Pat. No. 5,908,777, incorporated herein by reference). In another non-limiting example, the targeting ligand may comprise an integrin receptor ligand, described in U.S. Pat. No. 6,083,741, incorporated herein by reference.

Still further, a nucleic acid delivery vehicle may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated nucleic acid targeting vehicles include a cell receptor-specific ligand and a nucleic acid-binding agent. Others have a cell receptor-specific ligand to which the nucleic acid to be delivered has been operatively attached. Several ligands have been used for receptor-mediated nucleic acid transfer (EPO 0273085), which establishes the operability of the technique. In certain aspects, a ligand will be chosen to correspond to a receptor specifically expressed on the target cell population.

The targeting moiety can be directly linked to the polymer or indirectly linked through a spacer. Suitable targeting moieties include, but are not limited to peptide ligands, carbohydrates, lipids, polynucleotides, antibodies, aptamers, or combinations thereof. The antibody can be a fragment that is capable of binding the target polypeptide. Antibodies or antibody fragments can be single chained, humanized, chimeric, monoclonal, or polyclonal.

One embodiment provides a lipid-polymer conjugate having folic acid as the cell-targeting moiety. The folate receptor is, frequently overexpressed in cancer cells and is therefore of relevance for targeting tumors for gene delivery (Holm et al., 1999; Leaman and Low, 2001; Lu and Low, 2002; Ward, 2000). In addition, because of its importance to tumor cell biology, the interaction between folic acid and its receptor, as well as the intracellular trafficking dynamics of the receptor, are well characterized (Dauty, et al., Bioconjug. Chem., 13(4):831-9 (2002); Marchant, et al., J. Biol. Chem., 277(36):33325-33 (2002)). Importantly, it is known that retroviruses that bind the folate receptor are able to transduce cells, which suggests that the folate internalization pathway is not a ‘dead-end’ pathway for retroviruses (Viejo-Borbolla, et al., Virus Res., 108(1-2):45-55 (2005)). These viruses were pseudotyped with ecotropic envelope proteins fused with a single chain variable fragment (scFv) against the folate receptor at their N-termini. The modified viruses bound to the folate receptor and were able to infect cells via the wild-type ecotropic receptor about 10-fold less efficiently than unmodified viruses.

Additional targeting moieties include transferrin, RGD, Epidermal growth factor (EGF), Fibroblast growth factor (FGF), Tumor specific antibodies such as Herceptin, 0250, anti-Ep-CAM, CD34, SSCA-1, and CAM.

### III. Methods of Manufacture

Lipid-polymer conjugates can be created that contain a targeting group or a charged group (primary amine or carboxylic acid). The viruses may be modified with multiple types of lipid-polymer constructs: 1) lipid-polymer (high molecular weight) constructs that block non-specific binding, 2) lipid-polymer-targeting moiety constructs that promote specific binding to cells that express a target ligand or receptor, and 3) lipid-polymer-charged group (anionic or cationic) constructs that fine tune the balance between non-specific and specific virus binding.

The conjugates can be prepared using heterobifunctional protected polymers, for example PEGs, using standard procedures for conjugation to amine-functionalized PEG (Dube, et al., Bioconjug. Chem., 13(3):685-92 (2002); Leamon and Low, Proc. Natl. Acad. Sci. 88(13):5572-6 (1991); Leamon, et al., Bioconjug Chem., 10(6):947-57 (1999); Wang, et al., Bioconjug. Chem., 7(1):56-62 (1996)). Folic acid can be used as a targeting moiety. Since the coupling to either the α or γ acid site on folic acid yields a ligand with good affinity for its cognate receptor, it is not necessary to protect either group prior to the coupling reaction (Leamon and Reddy, Adv. Drug Deliv. Rev., 56(8):1127-41 (2004)). Fmoc-PEG-NHS can be coupled to a PE-amine derivative (Avanti Polar Lipids) followed by the removal of the Fmoc group to yield an amine. This amine can then be used to couple folate-NHS to yield PE-PEG-Folate. The activated folic acid will be prepared by standard carbodiimide-succinimide activation procedures (Hermanson, Bioconjugate Techniques; 1^{st} Ed., San Diego, Academic Press (1996)). Constructs that have a charged group on the distal end of the PEG, can be made rather than those having folic acid or another specific targeting moiety. The synthesis of charged PEGs can be accomplished by either leaving the carboxylic acid or primary amine site (which are charged at physiological pH) of PEG unreacted, or by coupling a new charged moiety to the available reactive site. If the weakly acidic and basic sites present on the PEG initially are not suitable for these studies, we will use simple chemistries to link sulfonates, phosphonates, and secondary or tertiary amines to the PEG end group.

### IV. Methods of Use

The disclosed lipid-polymer conjugates can be used to engineer the tropism of retroviruses. One method for engineering the tropism of retroviruses provides non-covalently modifying the surfaces of the viruses with functionalized lipid-PEG conjugates. The functionalized lipid-PEG conjugates enable the viruses to bind to cell-type specific receptors, while still maintaining the ability of the viruses to efficiently transfer genes to cells.

A. Gene Therapy

Embodiments of the present disclosure provide compositions and methods applicable for gene therapy protocols and the treatment of gene related diseases or disorders. Gene therapy now is becoming a viable alternative to various conventional therapies, especially in the area of cancer treatment. Limitations such as long term expression of transgenes and immuno-destruction of target cells through the expression of vector products, which have been said to limit the implementation of genetic therapies, are not concerns in cancer therapies, where destruction of cancer cells is desired.

It is important in gene transfer therapies, especially those involving treatment of cancer, to kill as many of the cells as quickly as possible. One goal of current cancer research is to find ways to improve the efficacy of one or more anti-cancer agents by combining such an agent with gene therapy. Thus, the use of “combination” therapies may be favored. Such combinations may include gene therapy and radiotherapy or chemotherapy. Gene therapy could be used similarly in conjunction with the nucleic acid delivery composition and/or other agents.

Another embodiment provides the use of multi-gene therapy. In this situation, more than one therapeutic gene would be transferred into a target cell. The genes could be from the same functional group (e.g., both tumor suppressors, both cytokines, etc.) or from different functional groups (e.g., a tumor suppressor and a cytokine). By presenting particular combinations of therapeutic genes to a target cell, it may be possible to augment the overall effect of either or both genes on the physiology of the target cell.

1. Inducers of Cellular Proliferation

In one embodiment, the virus in the nucleic acid delivery vehicle encodes an anti-sense mRNA directed to a particular inducer of cellular proliferation is used to prevent expression of the inducer of cellular proliferation. The proteins that induce cellular proliferation further fall into various categories dependent on function. The commonality of all of these proteins is their ability to regulate cellular proliferation. Representative oncogenes that can be targeted include, but are not limited to met, ret, ErB2/Her2/neu, ras, Bcl-2, sis, and c-myc.

2. Inhibitors of Cellular Proliferation

In certain embodiments, the restoration of the activity of an inhibitor of cellular proliferation through a genetic construct is contemplated. Tumor suppressor oncogenes function to inhibit excessive cellular proliferation. The inactivation of these genes destroys their inhibitory activity, resulting in unregulated proliferation. Representative genes that can be expressed to suppress oncogenes include, but are not limited to p53, p16, C-CAM, Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf; erb, fins, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

In certain embodiments, it is contemplated that genetic constructs that stimulate apoptosis will be used to promote the death of diseased or undesired tissue. Apoptosis, or programmed cell death, is an essential process for normal embryonic development, maintaining homeostasis in adult tissues, and suppressing carcinogenesis. The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli. The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

4. Diseases to be Treated

Cell dysfunction can also be treated or reduced using the disclosed compositions and methods. In particular, diseases amenable to gene therapy are specifically targeted. The disease can be in children, for example individuals less that 18 years of age, typically less than 12 years of age, or adults, for example individuals 18 years of age or more. Thus, embodiments of the present disclosure are directed to treating a host diagnosed with a disease, in particular a genetic disease, by introducing a vector into the host cell wherein the vector specifically binds to the cell type or cell state affected by the disease and wherein the vector comprises a nucleic acid encoding a therapeutic protein. In another embodiment, an inhibitory RNA is directed to a specific cell type or state to reduce or eliminate the expression of a protein, thereby achieving a therapeutic effect. The present disclosure encompasses manipulating, augmenting or replacing genes to treat diseases caused by genetic defects or abnormalities.

Suitable genetic based disease that can be treated with the compositions disclosed herein include but are not limited to:

Mitochondrial Disease:

Alpers Disease; Barth syndrome; β-oxidation defects; carnitine-acyl-carnitine deficiency; carnitine deficiency; co-enzyme Q10 deficiency; Complex I deficiency; Complex II deficiency; Complex III deficiency; Complex IV deficiency; Complex V deficiency; cytochrome c oxidase (COX) deficiency, LHON—Leber Hereditary Optic Neuropathy; MM—Mitochondrial Myopathy; LIMM—Lethal Infantile Mitochondrial Myopathy; MMC—Maternal Myopathy and Cardiomyopathy; NARP Neurogenic muscle weakness, Ataxia, and Retinitis Pigmentosa; Leigh Disease; FICP—Fatal Infantile Cardiomyopathy Plus, a MELAS-associated cardiomyopathy; MELAS—Mitochondrial Encephalomyopathy with Lactic Acidosis and Strokelike episodes; LDYT—Leber's hereditary optic neuropathy and Dystonia; MERRF—Myoclonic Epilepsy and Ragged Red Muscle Fibers; MHCM—Maternally inherited Hypertrophic CardioMyopathy; CPEO—Chronic Progressive External Ophthalmoplegia; KSS—Kearns Sayre Syndrome; DM—Diabetes Mellitus; DMDF Diabetes Mellitus+DeaFness; CIPO—Chronic Intestinal Pseudoobstruction with myopathy and Ophthalmoplegia; DEAF—Maternally inherited DEAFness or aminoglycoside-induced DEAFness; PEM—Progressive encephalopathy; SNHL—SensoriNeural Hearing Loss; Encephalomyopathy; Mitochondrial cytopathy; Dilated Cardiomyopathy; GER—Gastrointestinal Reflux; DEMCHO—Dementia and Chorea; AMDF—Ataxia, Myoclonus; Exercise Intolerance; ESOC Epilepsy, Strokes, Optic atrophy, & Cognitive decline; FBSN Familial Bilateral Striatal Necrosis; FSGS Focal Segmental Glomerulosclerosis; LIMM Lethal Infantile Mitochondrial Myopathy; MDM Myopathy and Diabetes Mellitus; MEPR Myoclonic Epilepsy and Psychomotor Regression; MERME MERRF/MELAS overlap disease; MHCM Maternally Inherited Hypertrophic CardioMyopathy; MICM Maternally Inherited Cardiomyopathy; MILS Maternally Inherited Leigh Syndrome; Mitochondrial Encephalocardiomyopathy; Multisystem Mitochondrial Disorder (myopathy, encephalopathy, blindness, hearing loss, peripheral neuropathy); NAION Nonarteritic Anterior Ischemic Optic Neuropathy; NIDDM Non-Insulin Dependent Diabetes Mellitus; PEM Progressive Encephalopathy; PME Progressive Myoclonus Epilepsy; RTT Rett Syndrome; SIDS Sudden Infant Death Syndrome; MIDD Maternally Inherited Diabetes and Deafness; and MODY Maturity-Onset Diabetes of the Young.

Nuclear Disease:

Muscular Dystrophies, Ellis-van Creveld syndrome, Marfan syndrome, Myotonic dystrophy, Spinal muscular atrophy, Achondroplasia, Amyotrophic lateral sclerosis, Charcot-Marie-Tooth syndrome, Cockayne syndrome, Diastrophic dysplasia, Duchenne muscular dystrophy, Ellis-van Creveld syndrome, Fibrodysplasia ossificans progressive, Alzheimer disease, Angelman syndrome, Epilepsy, Essential tremor, Fragile X syndrome, Friedreich's ataxia, Huntington disease, Niemann-Pick disease, Parkinson disease, Prader-Willi syndrome, Rett syndrome, Spinocerebellar atrophy, Williams syndrome, Ataxia telangiectasia, Anemia, sickle cell, Burkitt lymphoma, Gaucher disease, Hemophilia, Leukemia, Paroxysmal nocturnal hemoglobinuria, Porphyria, Thalassemia, Crohn's disease, Alpha-1-antitrypsin deficiency, Cystic fibrosis, Deafness, Pendred syndrome, Glaucoma, Gyrate atrophy of the choroid and retina, Adrenal hyperplasia, Adrenoleukodystrophy, Cockayne syndrome, Long QT syndrome, Immunodeficiency with hyper-IgM, Alport syndrome, Ellis-van Creveld syndrome, Fibrodysplasia ossificans progressive, Waardenburg syndrome, Werner syndrome.

Infectious Disease:

Viral—AIDS, AIDS Related Complex, Chickenpox (Varicella), Common cold, Cytomegalovirus Infection, Colorado tick fever, Dengue fever, Ebola haemorrhagic fever, Epidemic parotitis, Flu, Hand, foot and mouth disease, Hepatitis—Herpes simplex, Herpes zoster, HPV, Influenza, Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease—Yellow fever; Bacterial—Anthrax, Bacterial Meningitis, Brucellosis, Bubonic plague, Campylobacteriosis, Cat Scratch Disease, Cholera, Diphtheria, Epidemic Typhus, Gonorrhea, Hansen's Disease, Legionellosis, Leprosy, Leptospirosis, Listeriosis, Lyme Disease, Melioidosis, MRSA infection, Nocardiosis, Pertussis, Pneumococcal pneumonia, Psittacosis, Q fever, Rocky Mountain Spotted Fever or RMSF, Salmonellosis, Scarlet Fever, Shigellosis, Syphilis, Tetanus, Trachoma, Tuberculosis, Tularemia, Typhoid Fever, Typhus, Whooping Cough; Parasitic—African trypanosomiasis, Amebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amebic infection, Giardiasis, Gnathostomiasis, Hymenolepiasis, Isosporiasis, Kala-azar, Leishmaniasis, Malaria, Metagonimiasis, Myiasis, Onchocerciasis, Pediculosis, Pinworm Infection, Scabies, Schistosomiasis, Taeniasis, Toxocariasis, Toxoplasmosis, Trichinellosis, Trichinosis, Trichuriasis, Trypanosomiasis.

Cancers:

Breast and ovarian cancer, Burkitt lymphoma, Chronic myeloid leukemia, Colon cancer, Lung cancer, Malignant melanoma, Multiple endocrine neoplasia, Neurofibromatosis, p53 LieFrauMeni, Pancreatic cancer, Prostate cancer, retinoblastoma, von Hippel-Lindau syndrome, Polycystic kidney disease, Tuberous sclerosis.

Metabolic Disorders:

Adrenoleukodystrophy, Atherosclerosis, Best disease, Gaucher disease, Glucose galactose malabsorption, Gyrate atrophy, Juvenile onset diabetes, Obesity, Paroxysmal nocturnal hemoglobinuria, Phenylketonuria, Refsum disease, Tangier disease, Tay-Sachs disease, Adrenoleukodystrophy, Type 2 Diabetes, Gaucher disease, Hereditary hemochromatosis, Lesch-Nyhan syndrome, Maple syrup urine disease, Menkes syndrome, Niemann-Pick disease, Pancreatic cancer, Prader-Willi syndrome, Porphyria, Refsum disease, Tangier disease, Wilson's disease, Zellweger syndrome, progerias, SCID.

Autoimmune Disorders:

Autoimmune polyglandular syndrome, lupus, type I diabetes, scleroderma, multiple sclerosis, Crohn's disease, chronic active hepatitis, rheumatoid arthritis, Graves' disease, myasthenia gravis, myositis, antiphospholipid syndrome (APS), uveitis, polymyositis, Raynaud's phenomenon, and demyelinating neuropathies, and rare disorders such as polymyalgia rheumatica, temporal arteritis, Sjogren's syndrome, Bechet's disease, Churg-Strauss syndrome, and Takayasu's arteritis.

Inflammatory Disorders:

Alopecia, Diastrophic dysplasia, Ellis-van Creveld syndrome, Asthma, Arthritis, including osteoarthritis, rheumatoid arthritis, and spondyloarthropathies.

Age-Related Disorders:

Alzheimer Disease, Parkinson's Disease, Atherosclerosis, Age-Related Macular Degeneration, Age-related Osteoporosis.

The disclosed methods and compositions can also be used to treat, manage, or reduce symptoms associated with aging, in tissue regeneration/regenerative medicine, stem cell transplantation, inducing reversible genetic modifications, expressing inhibitory RNA, cognitive enhancement, performance enhancement, and cosmetic alterations to human or non-human animal.

5. Administration

The compositions provided herein may be administered in a physiologically acceptable carrier to cells or tissues grown outside of the body, in a culture dish (i.e., ex vivo). The compositions can be administered to cells or tissues that have been explanted from a patient for gene therapy applications, in which the cells, after they are genetically modified, will be implanted into the patient for a therapeutic effect. Alternatively, the compositions can be administered to primary cells, cell lines, or tissues that are being used for experimental, rather than therapeutic, purposes and therefore will not be re-implanted into a patient.

Alternatively, the compositions provided herein may be administered in a physiologically acceptable carrier to a host. Preferred methods of administration include systemic or direct administration to a cell. The compositions can be administered to a cell or patient, as is generally known in the art for gene therapy applications. In gene therapy applications, the compositions are introduced into a host in order to transfect specific cell types or cell states. “Gene therapy” includes both conventional gene therapy where a lasting effect is achieved by a single treatment, and the administration of gene therapeutic agents, which involves the one time or repeated administration of a therapeutically effective DNA or RNA.

The compositions can be combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween®, Pluronics® or PEG.

The compositions of the present disclosure can be administered parenterally. As used herein, “parenteral administration” is characterized by administering a pharmaceutical composition through a physical breach of a subject's tissue. Parenteral administration includes administering by injection, through a surgical incision, or through a tissue-penetrating non-surgical wound, and the like. In particular, parenteral administration includes subcutaneous, intraperitoneal, intravenous, intraarterial, intramuscular, intrasternal injection, and kidney dialytic infusion techniques.

Parenteral formulations can include the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Parenteral administration formulations include suspensions, solutions, emulsions in oily or aqueous vehicles, pastes, reconsitutable dry (i.e. powder or granular) formulations, and implantable sustained-release or biodegradable formulations. Such formulations may also include one or more additional ingredients including suspending, stabilizing, or dispersing agents. Parenteral formulations may be prepared, packaged, or sold in the form of a sterile injectable aqueous or oily suspension or solution. Parenteral formulations may also include dispersing agents, wetting agents, or suspending agents described herein. Methods for preparing these types of formulations are known. Sterile injectable formulations may be prepared using non-toxic parenterally-acceptable diluents or solvents, such as water, 1,3-butane diol, Ringer's solution, isotonic sodium chloride solution, and fixed oils such as synthetic monoglycerides or diglycerides. Other parentally-administrable formulations include microcrystalline forms, liposomal preparations, and biodegradable polymer systems. Compositions for sustained release or implantation may include pharmaceutically acceptable polymeric or hydrophobic materials such as emulsions, ion exchange resins, sparingly soluble polymers, and sparingly soluble salts.

Pharmaceutical compositions may be prepared, packaged, or sold in a buccal formulation. Such formulations may be in the form of tablets, powders, aerosols, atomized solutions, suspensions, or lozenges made using known methods, and may contain from about 0.1% to about 20% (w/w) active ingredient with the balance of the formulation containing an orally dissolvable or degradable composition and/or one or more additional ingredients as described herein. Preferably, powdered or aerosolized formulations have an average particle or droplet size ranging from about 0.1 nanometers to about 200 nanometers when dispersed.

As used herein, “additional ingredients” include one or more of the following: excipients, surface active agents, dispersing agents, inert diluents, granulating agents, disintegrating agents, binding agents, lubricating agents, sweetening agents, flavoring agents, coloring agents, preservatives, physiologically degradable compositions (e.g., gelatin), aqueous vehicles, aqueous solvents, oily vehicles and oily solvents, suspending agents, dispersing agents, wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other “additional ingredients” which may be included in the pharmaceutical compositions are known. Suitable additional ingredients are described in Remington's Pharmaceutical Sciences, Mack Publishing Co., Genaro, ed., Easton, Pa. (1985).

Dosages and desired concentrations modified vectors disclosed herein in pharmaceutical compositions of the present disclosure may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

B. Virus Detection

One embodiment provides a method for detecting or imaging a virus. The lipid-conjugates could be used to label viruses to enable them to be detected, quantified, or observed. This method could be used to detect viruses for any number of applications, including, for example, testing the quality of water supplies, for the diagnosis of human or animal or plant infectious diseases, or for detecting biowarfare viral agents.

The methods include mixing one or more of the disclosed lipid-polymer conjugates so that the lipid-polymer conjugate intercalates into the membrane of the virus. Typically, the lipid-polymer conjugate has a detectable label attached to the polymer instead of a targeting moiety. The detectable label can be a fluorescent tag, radioisotope, or any other detectable label known in the art. Fluorescent labels can include quantum dots or fluorescently labeled antibodies. Enzyme-labeled antibodies, such as horse radish peroxidase labeled antibodies, could be used for later detection in colorimetric or fluorescent substrate assays. Labels that alter the physico-chemical properties of the viruses to allow for more efficient separation or purification could be used. For example, biotin labels could be used to enable the viruses to be captured with streptavidin-coated beads.

C. Vaccines

One embodiment provides a vaccine in which the nucleic acid delivery vehicle includes a virus that has been decorated with one or more lipid-polymer conjugates that function to target the virus to bind to a specific cell type of the immune system, such as dendritic cells. Representative targeting moieties that could be incorporated into the lipid-polymer conjugate that would be useful in vaccines include, but are not limited to CD40, anti-CD4 antibodies or peptides, anti-CD8 antibodies or peptides, and anti-CD64 antibodies or peptides.

One embodiment provides a vaccine in which the nucleic acid delivery vehicle includes a virus that has been decorated with one or more lipid-polymer conjugates that function as adjuvants that stimulate a specific cell type of the immune system (such as dendritic cells) when the virus binds to the cell. Representative targeting moieties that could be incorporated into the lipid-polymer conjugate that would be useful in vaccines include, but are not limited to: CCL19 (a CC chemokine that binds to the chemokine receptor CCR7 which is expressed on mature dendritic cells (DC) and distinct T- and B-cell subpopulations), Flt3-ligand, and toll-like receptor ligands such as bacterial lipopolysaccharides, lipoproteins, and flagellin.

One embodiment provides a vaccine in which the nucleic acid delivery vehicle includes a virus encoding an immunogenic polypeptide. Representative polypeptides useful in vaccines include, but are not limited to: GMCSF, CCL20, IL-2, I1-4, IL-5, I1-6, IL-10, IL-12, IL-13, interferon gamma (IFN), viral antigens such as HIV-1 gp120, influenza envelope proteins such as RN and F, and tumor specific antigens such as SPAS-1, and melanoma associated antigen gp100. The manner of administration of a vaccine may be varied widely. Any of the conventional methods for administration of a vaccine are applicable. For example, a vaccine may be conventionally administered intravenously, intradermally, intraarterially, intraperitoneally, intralesionally, intracranially, intraarticularly, intraprostaticaly, intrapleurally, intratracheally, intranasally, intravitreally, intravaginally, intratumorally, intramuscularly, intraperitoneally, subcutaneously, intravesicularily, mucosally, intrapericardially, orally, rectally, nasally, topically, in eye drops, locally, using aerosol, injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

A vaccination schedule and dosages may be varied on a patient by patient basis, taking into account, for example, factors such as the weight and age of the patient, the type of disease being treated, the severity of the disease condition, previous or concurrent therapeutic interventions, the manner of administration and the like, which can be readily determined by one of ordinary skill in the art.

In many instances, it will be desirable to have multiple administrations of the vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters at intervals of 1-5 years, usually three years, will be desirable to maintain protective levels of the antibodies.

The course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays. Other immune assays can be performed and assays of protection from challenge with the antigen can be performed, following immunization.

### V. Kits

Another embodiment provides a kit. The kit includes a container suitable for shipping and housing additional elements of the kit. In a preferred embodiment the kit includes the disclosed lipid-polymer conjugates and instructions for modifying the surface of a virus. In some embodiments, the kit includes a virus encoding a predetermined polypeptide or nucleic acid. The kit also optionally includes additional reagents such as buffers optimally formulated for virus stability and lipid-conjugate incorporation, chromatography or spin columns or other precipitation reagents to separate free lipid-polymer conjugates and unlabeled viruses from labeled viruses, reagents and controls to quantify the extent to which the viruses have been modified by the lipid-conjugates or to quantify the activity of the virus of the extent to which they successfully genetically modify cells. The kit optionally includes a set of targeting molecules (e.g., antibodies or peptides) that, when mixed with virus particles that have been previously modified by the lipid-conjugates, would further modify the virus to enhances its ability to bind to a specific cellular receptor.

The kit optionally includes written directions for delivering the nucleic acid to a cell or host. A host includes a mammal, preferably a human. Typical cells include, but are not limited to eukaryotic cells, preferably mammalian cells, even more preferably human cells. Compositions that can be used to deliver nucleic acids to non-human cells may also be included.

### EXAMPLES

### Example 1

### Synthesis of Conjugates

A representative lipid-polymer conjugate includes an “anchor” lipid tail group for intercalation into the virus' phospholipid membrane. The lipid anchor is conjugated to a polymer such as PEG. The PEG can be of variable length, and has a functional terminus at the distal end (anchor-PEG-functional group architecture). Various different lipid anchor structures can be conjugated to a PEG spacer (2000 Dalton) having a biotin functional group (biotin). Phospholipid anchors with a 1,2-diacyl-sn-glycero-3-phosphatidyl ethanolamine (PE) structure are used, including anchors with two lipid tails (stearoyl and palmitoyl analogs), and a single-chain anchor (oleyl ether). PEG 5000 derivatives using heterobifunctional PEGs as linking agents between PE and biotin functionalities can also be used. For example, Fmoc-PEG-NHS (Nektar Therapeutics) can be used to couple the commercially available starting materials biotin-NH₂ or biotin-hydrazide to the activated acid (—NHS) end of the PEG. Deprotection of the amine terminus (-Fmoc protecting group) with piperazine will then allow for a second coupling reaction to take place, this time with PE-NHS (Avanti Polar Lipids), thus yielding a PE-PEG-Biotin construct. Similar syntheses are applicable to single fatty tail anchor derivatives. Given the extensive array of commercially available functionalized lipids and PEG chains, a wide range of structures will be available, should our initial constructs require further modification.

### Example 2

### Intercalation of Conjugates into Virus Surfaces

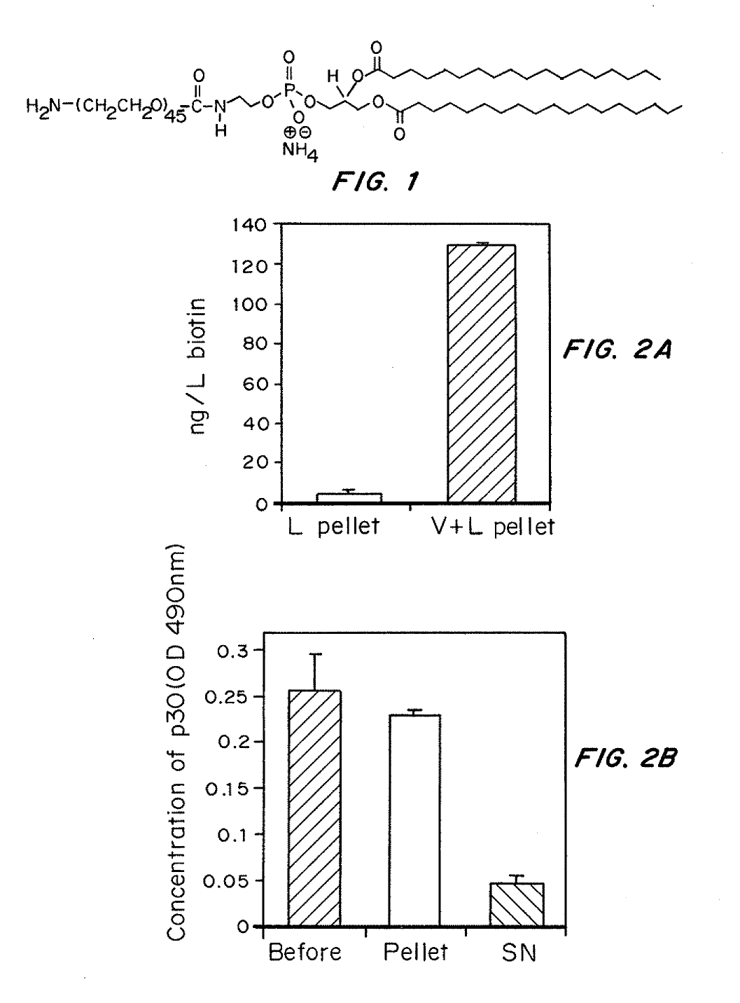

Stocks of lacZ amphotropic retrovirus, produced by TELCeB6-A cells, were brought to 6 μg/mL of DSPE-PEG-biotin conjugate, and incubated them for 2 hours at 4° C. to allow the lipid conjugates to incorporate into the virus particles. To separate retroviruses from unincorporated lipid conjugate, Polybrene, PB (320 μg/mL) was added to the mixtures. Polybrene causes the viruses to aggregate into large Polybrene-virus complexes and enables the viruses to be rapidly pelleted by low speed centrifugation. The viruses were pelleted by low speed centrifugation (4° C., 30 min, 10000×g), resuspended in TBS to their original volume, and the amount of biotin and virus (virus capsid protein, p30) in the pellets was quantified by ELISA. To account for the possibility that unincorporated lipid conjugate migrates with the polymer complexes, the lipid conjugate without the virus was pelleted (FIG. 2A). Significantly more biotin was detected in the pellet when virus was present as compared to the control and about 85% of virus was found in the pellet as compared to the stock (i.e., before Polybrene addition, FIG. 2B). These results show that the lipid conjugates were pelleted by centrifugation only when they were mixed with retroviruses, which suggests that they are physically associated with the viruses.

### Example 3

### Lipid-Conjugates Remain Stably Integrated Within The Lipid Bilayer Of Retroviruses

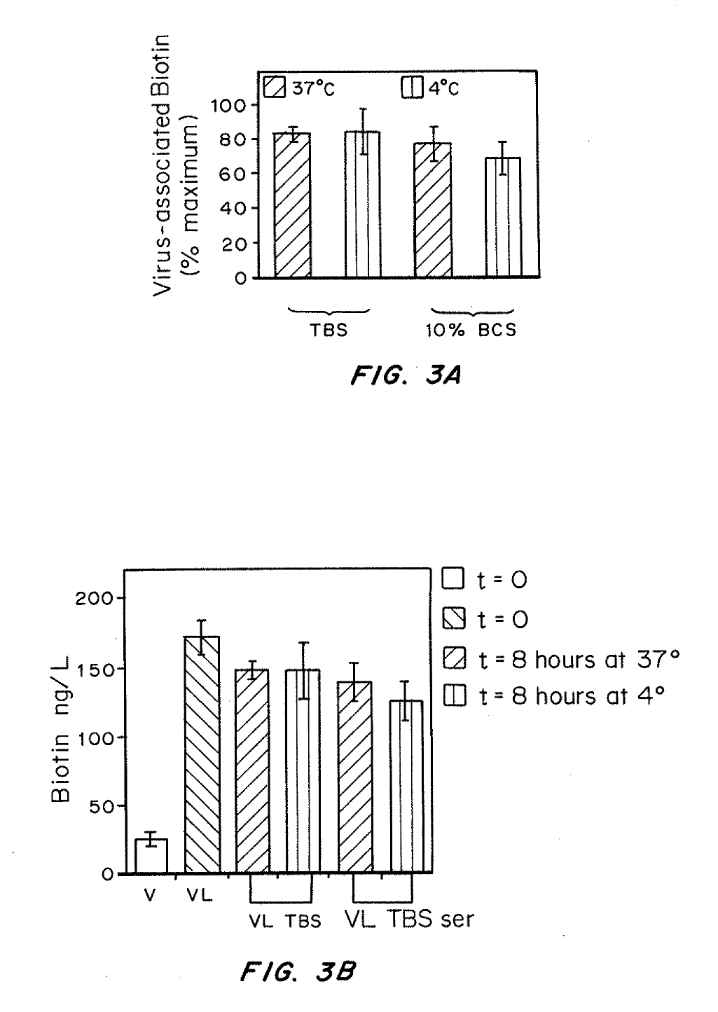

Amphotropic retrovirus was labeled with DSPE-PEG(2000)-biotin, separated from free lipid conjugate by centrifugation, resuspended in TBS w/or w/o 10% BCS, then incubated for 8 h at 4° C. (striped bars) or 37° C. (solid bars). The amount of DSPE-PEG(2000)-biotin conjugate associated with the particles was quantified by ELISA, and is reported as a percentage of the amount of biotin that associated with the particles at time zero (FIG. 3A). Studies with virus modified with the DSPE-PEG-biotin construct show that 70 to 80% of the constructs remain stably associated with the viruses after an 8 hour incubation time. The dissociation rate of the constructs does not appear to be affected by the temperature of incubation (4° C. versus 37° C.), and appears to be slightly accelerated by serum (10% BCS).

Thus to determine if the lipid conjugate remains stably associated with the virus particles, we resuspended the virus in a medium that did not contain any free conjugate and allowed the lipid to dissociate from the virus surface. We incubated a stock of retrovirus with lipid (6 μg/mL for 2 hours, 4° C.), added Polybrene (320 μg/mL) and centrifuged to pellet modified virus from unincorporated lipid conjugates. The pellet was resuspended in TBS or TBS containing 10% FBS and incubated at 4° C. or 37° C. for 8 hours. After the incubation, we separated lipid conjugate that may have eluted from the particles via Polybrene addition and subsequent centrifugation and quantified the amount of virus and biotin in the pellet by ELISA. We found that the amount of construct associated with the virus declined 30% E 5 after 8 hours (FIG. 3B). The dissociation rate was not significantly affected by temperature or in the presence of serum.

### Example 4

### Rate That The Lipid-Conjugates Anchor Within Retrovirus Membranes

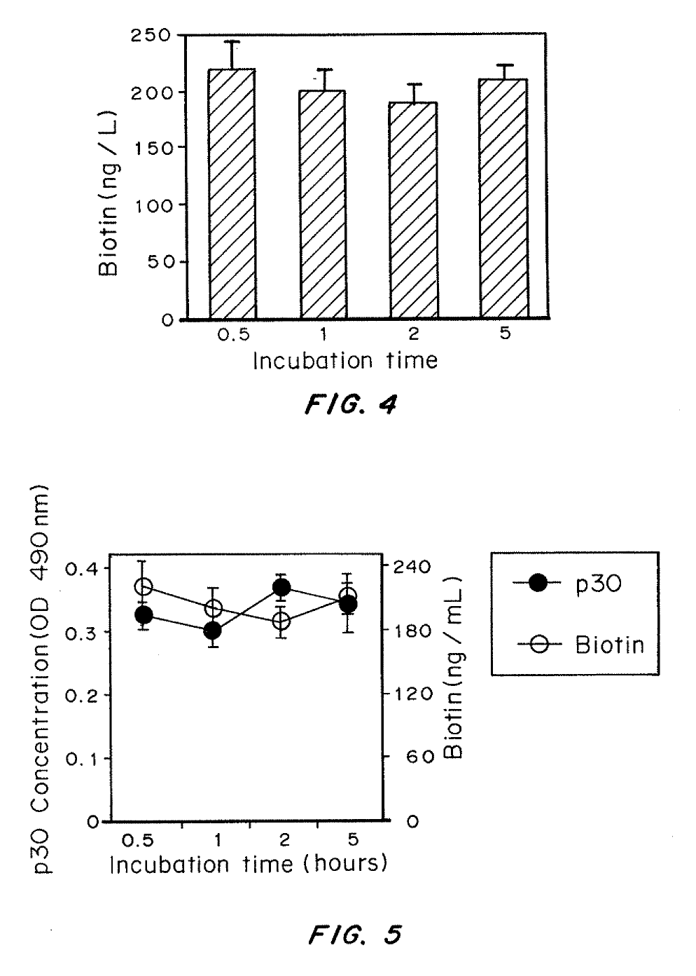

To examine the rate of DSPE-PEG-biotin conjugate anchoring, lentivirus was treated with 2 μM of DSPE-PEG-biotin for different periods of time at 37° C., pelleted by centrifugation, resuspended in PBS, and the amount of virus and biotin in the sample quantified by ELISA. The amount of conjugate that is incorporated into the viruses reaches a maximum level in less than 30 minutes (FIG. 4).

Retrovirus stock was concentrated 10-fold in TBS containing 6 μg/mL lipid conjugate, incubated at 4° C. for 0.5, 1, 2, 5 hours, separated by PB addition and centrifugation, and analyzed using an ELISA for p30 and biotin (FIG. 5). The amount of biotin incorporated into virus particles was the same at all time points, even when the virus was incubated with the conjugate for only 30 minutes. Thus the data suggest that the amount of conjugate attached to the virus reaches a maximum level in less than 30 minutes, much more rapidly than the rate of viral decay.

### Example 5

### Lipid Conjugate Co-Localize With The Virus Particles

To determine if the lipid conjugate co-localize with the virus particles rather than just co-migrating to the pellet upon centrifugation and to account for the possibility that the lipid conjugates may be associated with Polybrene complexes in the presence of virus but not directly associated with viruses, lipid modified GFP virus that had been incubated with fluorescently labeled streptavidin was visualized. Stocks of centrifuged lacZ amphotropic GFP-lentivirus were concentrated 10-fold in TBS that contained 6 μg/mL lipid-conjugate, and then incubated for 2 hours at 4° C. Lipid-modified or unmodified (control) GFP lentivirus incubated with 10 mM rhodamine-labeled streptavidin was visualized by epifluoresence microcopy. GFP-labeled virus particles modified with DSPE-PEG-biotin conjugate co-localized with rhodamine, whereas the GFP virus particles not modified did not co-localize with rhodamine.

### Example 6

### Binding Properties of Modified Virus

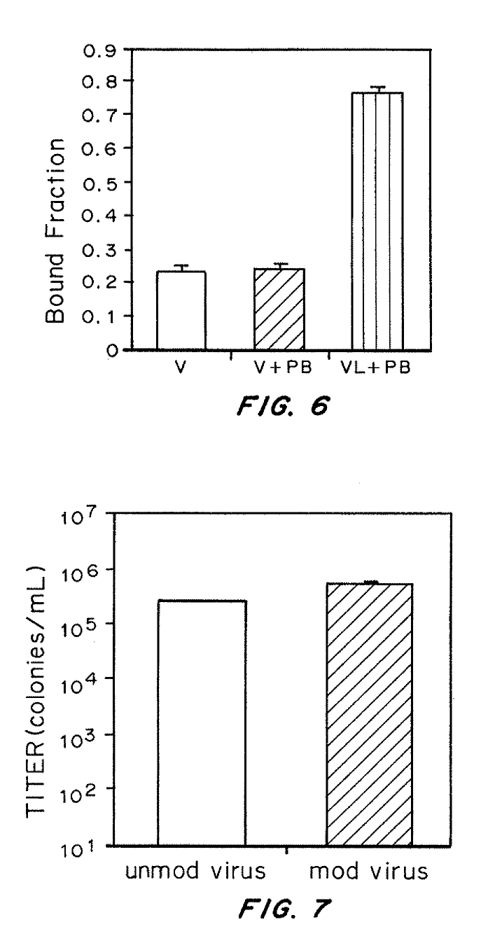

To determine if modified viruses have different binding properties than unmodified virus, 10-fold concentrated retrovirus particles were mixed with TBS containing lipid (6 μg/mL for 2 hours, 4° C.) or with TBS alone for the unmodified virus, then separated the virus from free lipid, and incubated samples on streptavidin coated plates. The samples were lysed, then quantified using a p30 ELISA and reported as the ratio of streptavidin-bound virus to the total amount of virus added to the streptavidin coated well (FIG. 6). The results show that modified viruses bound to streptavidin at a 3-fold higher level than unmodified viruses suggesting that the binding of modified viruses had been altered.

### Example 7

### Infectivity of Modified Viruses

To determine if lipid-modification affected the ability of the viruses to infect cells, the titer of modified virus particles was measured using an X-gal assay (FIG. 7). Modified lacZ retrovirus was separated from free conjugate by adding Polybrene followed by centrifugation. Hela cells that had been plated at 70,000 cells/well in a 12-well dish the day before were transduced using the modified virus. The titer for modified and unmodified virus stock was not statistically different suggesting that the modification did not reduce the infectivity of the virus particles.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

1. A nucleic acid delivery vehicle comprising:
(a) an enveloped virus; and
(b) a lipid-conjugated polymer intercalated into the envelope of the virus.

2. The nucleic acid delivery vehicle of claim 1, wherein the lipid-conjugated polymer comprises (1) a biocompatible polymer having first and second ends; (2) a multi-chain lipid conjugated to the first end; and (3) a targeting moiety conjugated to the second end.

3. The nucleic acid delivery vehicle of claim 1 wherein the virus encodes one or more polypeptides to be expressed in a target cell.

4. The nucleic acid delivery vehicle of claim 1, wherein the lipid-conjugated polymer comprises poly(ethylene glycol).

5. The nucleic acid delivery vehicle of claim 1, wherein the lipid-conjugated polymer comprises a double-chained lipid.

6. The nucleic acid delivery vehicle of claim 6, wherein the double-chained lipid comprises 1,2-distearoyl-sn-glycero-3-phosphoethanolamine-N-[methoxy(polyethylene glycol)-2000.

7. The nucleic acid delivery vehicle of claim 1, wherein the targeting moiety is selected from the group consisting of folic acid, RGD, Epidermal growth factor (EGF), Fibroblast growth factor (FGF), Tumor specific antibodies such as Herceptin, G250, anti-Ep-CAM, CD34, SSCA-1, and CAM.

8. A virus comprising a lipid-conjugated polymer intercalated into a membrane of the virus.

9. The virus of claim 8, wherein the lipid-conjugated polymer comprises poly(ethylene glycol).

10. The virus of claim 8, wherein the lipid-conjugated polymer comprises a double-chained lipid.

11. The virus of claim 10, wherein the double-chained lipid comprises 1,2-distearoyl-sn-glycero-3-phosphoethanolamine-N-[methoxy(polyethylene glycol)-2000.

12. The virus of claim 8, wherein the targeting moiety is selected from the group consisting of folic acid, RGD, Epidermal growth factor (EGF), Fibroblast growth factor (FGF), Tumor specific antibodies selected from the group consisting of Herceptin, G250, anti-Ep-CAM, CD34, SSCA-1, and CAM.

13. A method for delivering a polynucleotide to a cell comprising contacting the cell with the virus of claim 8.

14. A method for modifying the surface of a virus comprising:
combining the virus with a lipid-conjugated polymer, wherein the lipid-conjugate polymer comprises a targeting moiety and intercalates into a membrane of the virus.

15. A method for targeting a virus comprising:
combining the virus with a lipid-polymer conjugate wherein the lipid-polymer conjugate intercalates into a membrane of the virus and comprises:
a biocompatible polymer having first and second ends;
a multi-chain lipid conjugated to the first end; and
a targeting moiety conjugated to the second end.

16. The method of claim 14, wherein the lipid-conjugated polymer comprises poly(ethylene glycol).

17. The method of claim 14, wherein the lipid-conjugated polymer comprises a double-chained lipid.

18. The method of claim 17, wherein the double-chained lipid comprises 1,2-distearoyl-sn-glycero-3-phosphoethanolamine-N-[methoxy(polyethylene glycol)-2000.

19. The method of claim 14, wherein the targeting moiety is selected from the group consisting of folic acid, RGD, Epidermal growth factor (EGF), Fibroblast growth factor (FGF), Tumor specific antibodies selected from the group consisting of Herceptin, G250, anti-Ep-CAM, CD34, SSCA-1, and CAM.

20. (canceled)

21. A kit for delivering nucleic acids to a cell or host comprising:
a container;
a retrovirus housed in the container; and
a lipid-polymer conjugate housed in the container, wherein the lipid-polymer conjugate comprises:
a biocompatible polymer having first and second ends;
a multi-chain lipid conjugated to the first end; and
a targeting moiety conjugated to the second end.

22. The kit of claim 21, further comprising written instructions for delivering the retrovirus to a cell or host.
